(19) United States
(12) Patent Application Publication
(10) Pub. No.: US 2013/0195770 A1
(43) Pub. Date: Aug. 1, 2013

(54) COMPOSITION AND METHOD FOR REDUCING ATHEROSCLEROTIC LESIONS

(71) Applicant: DuPont Nutrition Bioscience ApS, Copenhagen (DK)

(72) Inventors: Marta Jadwiga Bauerek, Arhus N (DK); Bent Raungaard, Hojbjerg (DK); Leif Schauser, Arhus C (DK); Charlotte Horsmans Poulsen, Brabrand (DK); Jorn Borch Soe, Mundelstrup (DK)

(73) Assignee: DuPont Nutrition Bioscience ApS, Copenhagen (DK)

(21) Appl. No.: 13/774,826

(22) Filed: Feb. 22, 2013

Related U.S. Application Data
(63) Continuation-in-part of application No. 13/696,271, filed on Nov. 5, 2012.
(60) Provisional application No. 61/376,023, filed on Aug. 23, 2010.

(30) Foreign Application Priority Data
May 7, 2010 (GB) 1007668.5

(51) Int. Cl. A61K 31/575; A23L 1/30; A61K 38/45

(52) U.S. Cl. CPC A61K 31/575; A61K 38/45; A23L 1/3008
USPC 424/48; 514/182; 552/544; 424/94.5

(57) ABSTRACT

The present invention relates to a method for reducing cholesterol absorption and the occurrence of atherosclerotic lesions in an animal comprising administering to the animal a composition comprising an effective amount of at least one cholesterol ester.

### RELATED APPLICATION DATA

This application is a continuation-in-part of U.S. patent application Ser. No. 13/696,271 filed on Nov. 5, 2012, which is a national phase of PCT App. No. IB2011/52009, filed on May 6, 2011, which claims priority to U.S. Provisional App. No. 61/376,023, filed Aug. 23, 2010, and GB App. No. 1007668.5, filed May 7, 2010.

### FIELD OF THE INVENTION

The described embodiments of a composition and method relate to reducing atherosclerotic lesions in an individual. More specifically the embodiments relate to reducing atherosclerotic lesions by administering an effective amount of cholesterol ester to the individual. The embodiments also relate to pharmaceutical compositions and foodstuffs comprising cholesterol esters.

### BACKGROUND

Cholesterol (CL) is an extremely important biological molecule of vital importance for mammalian cell structure and function. It is a major structural component of cell membranes, modulating membrane fluidity and essential for maintaining membrane integrity and permeability. Cholesterol is also a precursor for the synthesis of other steroids, including bile acids, vitamin D and steroid hormones (glucocorticoids, estrogens, progesterone, androgens and aldosterone). Additionally, cholesterol contributes to the development and functions of the central nervous system and it has essential functions in signal transduction, sperm development, and embryogenesis.

Cholesterol is the most abundant steroid in animal tissues and in the intestinal lumen. It is poorly soluble in an aqueous environment. As shown in Formula I, cholesterol includes four rings having trans-ring junctions, and the side chain and two methyl groups (C-18 and C-19) are at an angle to the rings above the plane with stereochemistry (as well as the hydroxyl group on C-3 also). Furthermore, there is a double bond between carbons 5 and 6. Thus, the molecule has a rigid planar four-ring nucleus with a flexible tail.

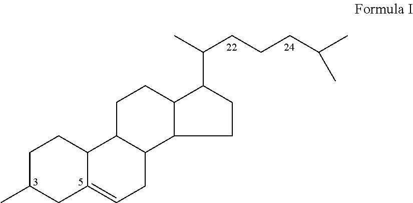 [SMILES: CC(C)CCCC(C)C1CCC2C1CCC1C3CCC(C)CC3=CCC12]

Within the human body cholesterol is transported through blood circulation in lipoprotein particles. Lipoproteins are aggregates of specific proteins (apolipoproteins (Apo-)) and various lipids. Lipoproteins have been divided into four major classes including chylomicrons, very-low density lipoproteins (VLDL), high-density lipoproteins, and low-density lipoproteins (LDL). LDLs consist mainly of cholesterol and cholesteryl esters as described below. The primary function of LDL particles is the transport of cholesterol to peripheral tissues, although a high level of LDL-cholesterol (LDL-C) in the blood plasma is associated with the development of atherosclerosis and atherosclerotic lesions. Atherosclerosis is a complex, pathological, inflammatory disorder characterized by progressive thickening of the arterial walls. Accumulation and oxidation of low-density lipoproteins (LDL) in the arterial wall causes multiple endothelial injuries and triggers a complex of biochemical, immune-modulatory and inflammatory reactions involving a range of different molecules and cell types. Those reactions lead to the development of atherosclerotic plaques.

Clinical studies show that plant sterols induce reduction in serum LDL-cholesterol concentrations in mild hypercholesterolemic subjects. Therefore dietary plant sterols are recommended as adjunctive lifestyle treatment for hypercholesterolemia While the absorption of intraluminal cholesterol and plant sterols is well described, the effects of dietary cholesteryl esters has not been considered. The present inventors have undertaken research to identify the effect of cholesterol esters on the levels of cholesterol in the blood.

### SUMMARY

The description of embodiments of a composition and method herein is based on the highly surprising discovery by the inventors that the presence of cholesterol esters can reduce the absorption of cholesterol by intestinal cells resulting in lower levels of blood cholesterol.

Therefore, according to a first aspect of the described embodiments there is provided a method for reducing cholesterol absorption in an animal comprising administering to the animal a composition comprising an effective amount of at least one cholesterol ester.

According to a second aspect of the described embodiments there is provided a method for reducing cholesterol absorption in an animal comprising administering to the animal a composition comprising at least one lipid acyltransferase.

According to a third aspect of the described embodiments there is provided a composition comprising at least one cholesterol ester for use in therapy.

According to a fourth aspect of the described embodiments there is provided a composition comprising at least one cholesterol ester for use in reducing the level of blood cholesterol in an individual.

According to a fifth aspect of the described embodiments there is provided a composition comprising at least one lipid acyltransferase for use in reducing the level of blood cholesterol in an individual.

According to a sixth aspect of the described embodiments there is provided a pharmaceutical composition comprising at least one cholesterol ester and at least one pharmaceutically acceptable diluent, excipient and/or carrier.

According to a seventh aspect of the described embodiments there is provided a foodstuff comprising at least one exogenously produced cholesterol ester.

According to an eighth aspect of the described embodiments there is provided the use of a cholesterol ester in the manufacture of a medicament for reducing the level of blood cholesterol in an individual.

According to an ninth aspect of the described embodiments there is provided the use of a lipid acyltransferase in the manufacture of a medicament for reducing the level of blood cholesterol in an individual.

According to a tenth aspect of the described embodiments there is provided a method for regulating cholesterol absorption in an individual not suffering from hypercholesterolemia comprising administering to the individual a composition comprising an effective amount of at least one cholesterol ester.

According to an eleventh aspect of the described embodiments there is provided a method for regulating cholesterol absorption in an individual not suffering from hypercholesterolemia comprising administering to the individual a composition comprising an effective amount of at least one lipid acyltransferase.

According to a twelfth aspect of the described embodiments there is provided a method for reducing atherosclerotic lesions in an individual comprising administering to the individual a composition comprising an effective amount of at least one cholesterol ester.

According to a thirteenth aspect of the described embodiments there is provided a composition for reducing atherosclerotic lesions in an individual comprising an effective amount of at least one cholesterol ester.

According to a fourteenth aspect of the described embodiments there is provided a pharmaceutical composition for reducing atherosclerotic lesions in an individual comprising an effective amount of at least one cholesterol ester and at least one of a pharmaceutically acceptable excipient, diluent, or carrier.

### BRIEF DESCRIPTION OF THE DRAWINGS

FIG. 1 shows sterol absorption into CaCo-2 cells using artificial micelles;

FIG. 2 shows intestinal absorption of sterols in mice using the plasma dual isotope ratio method;

FIG. 3 shows digital images of exemplary arteries examined;

FIG. 4 shows experimental results regarding observed areas of atherosclerotic plaque and lesions.

### DETAILED DESCRIPTION

In the description which follows, it will be understood that any of the features described are applicable to any aspect of the described embodiments unless explicitly stated otherwise.

In the human body, there are two major sources of cholesterol available to peripheral cells: the hepatic pool, i.e. the liver, which is the major center of cholesterol synthesis (although also other tissues are able to synthesize cholesterol), and the intraluminal pool, i.e. gastrointestinal tract with cholesterol derived from dietary origin, bile and desquamated intestinal epithelium. The average intake of cholesterol in Western diet is approximately 300-500 mg daily. Biliary secretions provide 800-1200 mg cholesterol daily, while the turnover of intestinal mucosal epithelium provides roughly 300 mg of cholesterol per day. The non-absorbed cholesterol is excreted with feces. The term ‘Intestinal absorption of cholesterol’ defines the transfer of intraluminal cholesterol into intestinal or thoracic duct lymph and it comprises of three steps: (1) intraluminal phase (hydrolysis of dietary lipids and micellization of cholesterol), (2) transport across the apical membrane of absorptive enterocytes (release of cholesterol from micelles and uptake into enterocytes), (3) intracellular phase (re-esterification and mobilization into chylomicrons followed by secretion into lymph and blood through the basolateral membrane of erythrocytes)

Cholesterol Esters

It will be apparent to the skilled person that as used herein, the term cholesterol ester relates to any cholesterol ester, for example, cholesterol fatty acid esters including cholesterol fatty acid esters in which the fatty acid is saturated or unsaturated. Furthermore, the terms cholesterol ester and cholesteryl ester are used interchangeably.

In various embodiments the cholesterol ester has a structure as shown in Formula II:

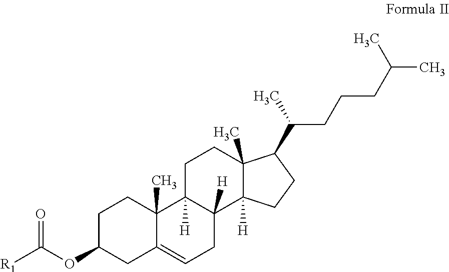

In one example aspect, R₁ in Formula II is a C1-C35 hydrocarbon group. Here the term “hydrocarbon” means any one of an alkyl group, an alkenyl group, or an alkynyl group, which groups may be linear, branched or cyclic, or an aryl group. The term hydrocarbon also includes those groups but wherein they have been optionally substituted. If the hydrocarbon is a branched structure having substituent(s) thereon, then the substitution may be on either the hydrocarbon backbone or on the branch; alternatively the substitutions may be on the hydrocarbon backbone and on the branch.

Suitable substituent(s) are hydroxyl groups. In various embodiments, the compound has between 0 to 3 substituents, or 0 to 2, or 0 or 1.

R₁ in Formula II may be a C4-C24 hydrocarbon group. R₁ may also be a C10-C23 hydrocarbon group, or a C9-C17 hydrocarbon group, such as a C13-C17 group. In one embodiment, R₁ is a C17 hydrocarbon group.

In one aspect, R₁ is a hydrocarbon group comprising an alkenyl group. In one example this hydrocarbon group comprises from 1 to 6 C═C double bonds. In another embodiment this hydrocarbon group comprising an alkenyl group comprises from 1 to 3 C═C double bonds.

In one aspect, R₁ is a saturated hydrocarbon group. R₁ may be a (CH₂)ₙCH₃ group, wherein n is zero or a positive integer. In various embodiments, n is an integer from 6 to 28, or 8 to 22, or 14 to 20, such as 14 to 18. In another embodiment n is 16.

In one embodiment, the cholesterol ester for use in the described embodiments comprises at least one fatty acid having a carbon chain length of 10:0, 10:1, 12:0, 12:1, 13:0, 13:1, 14:0, 14:1, 15:0, 15:1, 16:0, 16:1, 17:0, 18:0, 18:1, 18:2, 20:0, 20:1, 20:2 wherein the first number relates to the fatty acid carbon chain length and the second number refers to the number of double bonds present in the carbon chain.

In another embodiment, the cholesterol ester comprises at least one of cholesteryl linoleate (C18:2), cholesteryl oleate (C18:1), cholesteryl stearate (C18:0), cholesteryl palmitate(C16:0), cholesteryl palmitoleate (C16:1). cholesteryl myristate (C14:0), cholesteryl laurate (C12:0) and cholesteryl caprate (C10:0). In yet another embodiment, the cholesterol ester comprises a mixture of at least two of the recited esters.

It will be understood that the cholesterol ester may be obtained from any suitable source, naturally occurring or synthetic. In one embodiment, the cholesterol ester is enzymatically produced using a lipid acyltransferase. The cholesterol ester may be produced from at least one of egg, milk and/or meat.

It will be apparent to the skilled person that the cholesterol moiety of the cholesterol ester may be from any suitable source. In one example, the cholesterol moiety is from an animal source.

It will be understood that the sterol moiety for use in the methods and uses of the described embodiments is not a plant sterol.

It will further be apparent to the skilled person that when the cholesterol ester is a cholesterol fatty acid ester, the fatty acid can be provided from any suitable source. In one embodiment, the fatty acid is provided by a triglyceride or phospholipid.

In one embodiment, the hydrocarbon group is provided by a plant or animal source. In one embodiment, the hydrocarbon group is from dairy fat. In another embodiment, the hydrocarbon group is from at least one plant oil.

In a further embodiment, the hydrocarbon group is not provided from a plant source.

In one embodiment, the cholesterol ester is present in a foodstuff.

The foodstuff may be selected from one or more of: eggs, egg-based products, including but not limited to mayonnaise, salad dressings, sauces, ice creams, egg powder, modified egg yolk and products made therefrom; baked goods, including breads, cakes, sweet dough products, laminated doughs, liquid batters, muffins, doughnuts, biscuits, crackers and cookies; confectionery, including chocolate, candies, caramels, halawa, gums, including sugar free and sugar sweetened gums, bubble gum, soft bubble gum, chewing gum and puddings; frozen products including sorbets, frozen dairy products, including ice cream and ice milk; dairy products, including cheese, butter, milk, coffee cream, whipped cream, custard cream, milk drinks and yoghurts; mousses, whipped vegetable creams, meat products, including processed meat products; edible oils and fats, aerated and non-aerated whipped products, oil-in-water emulsions, water-in-oil emulsions, margarine, shortening and spreads including low fat and very low fat spreads; dressings, mayonnaise, dips, cream based sauces, cream based soups, beverages, spice emulsions and sauces. In certain examples, the foodstuff is milk or a milk product.

It will be apparent to the skilled person that the cholesterol ester may be produced exogenously and added to the foodstuff and/or may be produced in situ by the action of a lipid acyltransferase. It will be further apparent that a number of foodstuffs may comprise naturally occurring cholesterol esters. However, the skilled person would understand that these naturally occurring esters are present at sub-clinical levels which have no significant effect on the absorption of cholesterol by intestinal cells. The skilled person will understand that the described embodiments relate to methods and compositions which comprise additional amounts of cholesterol ester beyond that which may naturally present in a foodstuff.

It will be understood that the terms produced exogenously and exogenously produced as used herein means that the lipid acyltransferase is not produced in situ, for example, in the foodstuff, but is added thereto in an appropriate amount to give the desired concentration.

The skilled person will understand that cholesterol absorption occurs primarily in the duodenum and proximal jejunum at levels of efficiency that vary greatly among different individuals. There are two main phases of cholesterol absorption, the first takes place in the lumen and involves digestion and hydrolysis of dietary lipids followed by solubilization of cholesterol in mixed micelles containing bile acid and phospholipids. This solubilization facilitates the movement of cholesterol from the bulk phase of the lumen to the surface of the enterocyte. In the second phase, cholesterol crosses the mucosal cell membrane.

As used herein the terms blood cholesterol refers to the total cholesterol level in the blood. It will be apparent to the skilled person that this includes LDL cholesterol and HDL cholesterol and VLDL cholesterol.

It will be understood that the term animal used herein refers to both humans and other types of animal, particularly mammals. It will be further understood that as used herein the term individual refers to a human or other animal.

In one embodiment, the animal to be administered the compositions is a human. In another embodiment, the animal is a human suffering from hypercholesterolemia.

In one embodiment the methods relate to methods of treating hypercholesterolemia.

It will be understood that as used herein the term treating hypercholesterolemia refers to both treating individuals suffering from hypercholesterolemia and the prophylactic treatment of individuals at risk of developing hypercholesterolemia.

It will be apparent to a skilled person that individuals may suffer from different levels of hypercholesterolemia. In one embodiment an individual to be administered the compositions is an individual suffering from mild to moderate hypercholesterolemia (5.2-8.0 mmol cholesterol/L blood; LDL-C in the range from about 130-159 mg/dl [mild] to about 160-219 mg/dl [moderate]).

In a further embodiment the animal or individual is an animal or individual suffering from severe hypercholesterolemia (LDL-C of greater than 220 mg/dl).

In an alternative embodiment the animal or individual is an animal or individual having a LDL-C level of less than 130 mg/d1.

In a further aspect the described embodiments relate to a method for regulating cholesterol absorption in an individual not suffering from hypercholesterolemia comprising administering to said individual a composition comprising at least one cholesterol ester and/or at least one lipid acyltransferase.

It will be understood that administration of at least one cholesterol ester or lipid acyltransferase to an individual having normal levels of blood cholesterol may reduce or prevent an increase in the level of blood cholesterol.

It will be understood that the composition may be administered in any suitable form. The composition may be suitable for oral administration. The composition may be a foodstuff. In an alternative embodiment the composition is an oral composition suitable to be taken as a food supplement.

It will be understood that an individual may be at risk of developing hypercholesterolemia due to a variety of reasons, for example, as a result of obesity, diet, familial hypercholesterolemia, type 2 diabetes, hypothyroidism, or side effects of other medication.

In a preferred aspect the described embodiments relate to a composition comprising at least one cholesterol ester for use in therapy. More preferably, the composition further comprises at least one lipid acyltransferase.

in a further aspect, the described embodiments relate to a foodstuff comprising at least one exogenously produced cholesterol ester.

In one embodiment, the foodstuff further comprises at least one lipid acyltransferase. The skilled person will understand that if the foodstuff comprises a lipid acyl donor and cholesterol, further cholesterol esters as described above may be produced in situ in the foodstuff.

The foodstuff may be selected from one or more of: eggs, egg-based products, including but not limited to mayonnaise, salad dressings, sauces, ice creams, egg powder, modified egg yolk and products made there from; baked goods, including breads, cakes, sweet dough products, laminated doughs, liquid batters, muffins, doughnuts, biscuits, crackers and cookies; confectionery, including chocolate, candies, caramels, halawa, gums, including sugar free and sugar sweetened gums, bubble gum, soft bubble gum, chewing gum and puddings; frozen products including sorbets, frozen dairy products, including ice cream and ice milk; dairy products, including cheese, butter, milk, coffee cream, whipped cream, custard cream, milk drinks and yoghurts; mousses, whipped vegetable creams, meat products, including processed meat products; edible oils and fats, aerated and non-aerated whipped products, oil-in-water emulsions, water-in-oil emulsions, margarine, shortening and spreads including low fat and very low fat spreads; dressings, mayonnaise, dips, cream based sauces, cream based soups, beverages, spice emulsions and sauces.

In certain embodiments, the foodstuff is milk or a milk based product.

It will be apparent to the skilled person that in the described embodiments, the cholesterol ester is added to the foodstuff. In alternative embodiments the cholesterol ester is not added to the foodstuff. In further embodiments, the cholesterol ester is generated in the foodstuff. In yet further embodiments, the cholesterol is not generated in the foodstuff

The described embodiments also provide a pharmaceutical composition comprising at least one cholesterol ester and/or at least one lipid acyltransferase for use in the methods or uses of the present embodiments and a pharmaceutically acceptable carrier, diluent or excipient (including combinations thereof).

The pharmaceutical compositions may be for human or animal usage in human and veterinary medicine and will typically comprise any one or more of a pharmaceutically acceptable diluent, carrier, or excipient. Acceptable carriers or diluents for therapeutic use are well known in the pharmaceutical art, and are described, for example, in Remington's Pharmaceutical Sciences, Mack Publishing Co. (A. R. Gennaro edit. 1985). The choice of pharmaceutical carrier, excipient or diluent can be selected with regard to the intended route of administration and standard pharmaceutical practice. The pharmaceutical compositions may comprise as—or in addition to—the carrier, excipient or diluent any suitable binder(s), lubricant(s), suspending agent(s), coating agent(s), solubilizing agent(s).

Preservatives, stabilizers, dyes and even flavoring agents may be provided in the pharmaceutical composition. Examples of preservatives include sodium benzoate, sorbic acid and esters of p-hydroxybenzoic acid. Antioxidants and suspending agents may be also used.

There may be different composition/formulation requirements dependent on the different delivery systems. By way of example, the pharmaceutical composition of the described embodiments may be formulated to be delivered using a mini-pump or by a mucosal route, for example, as a nasal spray or aerosol for inhalation or ingestible solution, or parenterally in which the composition is formulated by an injectable form, for delivery, by, for example, an intravenous, intramuscular or subcutaneous route. Alternatively, the formulation may be designed to be delivered by both routes.

Where the agent is to be delivered mucosally through the gastrointestinal mucosa, it should be able to remain stable during transit though the gastrointestinal tract; for example, it should be resistant to proteolytic degradation, stable at acid pH and resistant to the detergent effects of bile.

Where appropriate, the pharmaceutical compositions can be administered by inhalation, in the form of a suppository or pessary, topically in the form of a lotion, solution, cream, ointment or dusting powder, by use of a skin patch, orally in the form of tablets containing excipients such as starch or lactose, or in capsules or ovules either alone or in admixture with excipients, or in the form of elixirs, solutions or suspensions containing flavoring or coloring agents, or they can be injected parenterally, for example intravenously, intramuscularly or subcutaneously. For parenteral administration, the compositions may be best used in the form of a sterile aqueous solution which may contain other substances, for example enough salts or monosaccharides to make the solution isotonic with blood. For buccal or sublingual administration the compositions may be administered in the form of tablets or lozenges which can be formulated in a conventional manner.

The pharmaceutical composition may be in a form that is suitable for oral delivery.

The cholesterol ester may be provided at a level in the foodstuff or pharmaceutical composition or food supplement to result in administration to an individual of a dosage of between about 0.001 g and about 10 g per day. about 0.01 g and about 5 g per day, about 0.1 g and about 3 g per day based on a recommended portion size in relation to food or a recommended dosage regime in relation to a pharmaceutical or food supplement.

In an alternative embodiment the cholesterol ester is provided at a dosage of less than 10 g per day, less than 7 g per day, less than 5 g per day, less than 3 g per day, less than 2 g per day, less than 1 g per day, less than 0.5 g per day, less than 0.1 g per day, less than 0.05 g per day or less than 0.01 g per day.

In an alternative embodiment the cholesterol ester is provided at a dosage of more than 0.01 g per day, more than 0.05 g per day, more than 0.1 g per day, more than 0.5 g per day, more than 1 g per day, more than 2 g per day, more than 3 g per day, more than 5 g per day, more than 7 g per day or more than 10 g per day.

In some embodiments the pharmaceutical composition or food supplement is administered before, or during, or after a meal. It will be understood that the terms before and after may mean within 2 hours, or 1 hour, or 30 minutes, or 15 minutes of beginning/finishing the meal. It will be understood that in some embodiments the meal may be a high cholesterol meal.

In an alternative embodiment the pharmaceutical or food supplement is designed to be taken 1, or 2, or 3 or 4 times daily.

In one embodiment, the foodstuff or pharmaceutical or food supplement comprises at least two different cholesterol esters.

Lipid Acyltransferases

It will be apparent to the skilled person that the at least one lipid acyltransferase for use in the aspects of the described embodiments may be any lipid acyltransferase.

For instance, the lipid acyltransferase for use in the described embodiments may be one as described in WO2004/064537, WO2004/064987, WO20Q5/066347, WO2006/008508 or WO2008/090395. These documents are incorporated herein by reference.

The lipid acyltransferase for use in any one of the methods and/or uses of the described embodiments may be a natural lipid acyltransferase or a variant lipid acyltransferase.

The term “lipid acyltransferase” as used herein means an enzyme which has acyltransferase activity (for example an enzyme classified as E.G. 2.3.1.x, in particular 2.3.1.43 in accordance with the Enzyme Nomenclature Recommendations (1992) of the Nomenclature Committee of the International Union of Biochemistry and Molecular Biology), whereby the enzyme is capable of transferring an acyl group from a lipid to cholesterol.

Suitably the lipid acyltransferase is one classified under the Enzyme Nomenclature classification (E.G. 2.3.1.43). Such enzymes are commercially available from DuPont Nutrition Bioscience ApS and are sold under the trade name LysoMax Oil™ and Food-Pro Cleanline™.

The lipid acyltransferase for use in any one of the methods and/or uses of the described embodiments may be a lipid acyltransferase that is capable of transferring an acyl group from a phospholipid to a sterol.

Other acyltransferases suitable for use in the described embodiments include phospholipidrdiacylglycerol acyltransferases from enzyme class E.0 2.3.1.158 as disclosed in WO03/100044 (incorporated herein by reference), diacyiglycerol-sterol O-acyltransferases from class E.0 2.3.1.73 which catalyse the reaction 1.2-diacyl-.w-glycerol+sterol→monoacylglycerol+sterol ester, and sterol -acyltransferases from class E.0 2.3.1.26 which catalyze the reaction acyl-CoA+cholesterol→CoA+cholesterol ester.

Suitably, the acyltransferase activity of enzymes for use in the described embodiments accounts for at least 5%, or at least 10%, or at least 20%, or at least 30%, or at least 40%, or 50%>, or at least 60% o, or at least 70%), or at least 80%, or at least 90% or at least 98% of the total enzyme activity. The % transferase activity (i.e. the transferase activity as a percentage of the total enzymatic activity) may be determined by the following protocol:

Determination of Lipid Acyltransferase Activity

Substrate: 50 mg Cholesterol (Sigma C8503) and 450 mg Soya phosphatidylcholine(PC), Avanti #441601 is dissolved in chloroform, and chloroform is evaporated at 4( )° C. under vacuum.

300 mg PC:cholesterol 9:1 is dispersed at 40° C. in 10 ml 50 mM HEPES buffer pH 7.

Enzymation:

250 μl substrate is added in a glass with lid at 40° C.

25 μl enzyme solution is added and incubated during agitation for 10 minutes at 40° C. The enzyme added should esterify 2-5% of the cholesterol in the assay.

Also a blank with 25 μl water instead of enzyme solution is analyzed.

After 10 minutes 5 ml Hexan:Isopropanol 3:2 is added.

The amount of cholesterol ester may be analyzed by HPLC using Cholesteryl stearate (Sigma C3549) standard for calibration.

Transferase activity is calculated as the amount of cholesterol ester formation per minute under assay conditions.

One Transferase Unit (TrU) is defined as μmol cholesterol ester produced per minute at 40° C. and pH 7 in accordance with the transferase assay given above.

The lipid acyltransferase used in the method and uses of the described embodiments may have a specific transferase unit (TrU) per mg enzyme of at least 25 TrU/mg enzyme protein.

Suitably the lipid acyltransferase for use in the described embodiments may be dosed in amount of 0.05 to 50 TrU per g phospholipid composition, suitably in an amount of 0.5 to 5 TrU per g phospholipid composition.

The enzymes suitable for use in the methods and/or uses of the described embodiments may have lipid acyltransferase activity as defined by the protocol below:

Protocol for the Determination of % Acyltransferase Activity:

A foodstuff to which a lipid acyltransferase for use according to the described embodiments has been added may be extracted following the enzymatic reaction with CHCl₃:CH₃OH 2:1 and the organic phase containing the lipid material is isolated and analyzed by GLC according to the procedure detailed herein below. From the GLC analysis or HPLC analysis the amount of free fatty acids and one or more cholesterol esters is determined. A control foodstuff to which no enzyme according to the described embodiments has been added, is analyzed in the same way.

Calculation:

From the results of the GLC (and optionally HPLC analyses) the increase in free fatty acids and cholesterol esters can be calculated:

Δ % fatty acid=% Fatty acid(enzyme)−% fatty acid(control);

Mv fatty acid=average molecular weight of the fatty acids;

Δ=Δ % cholesterol ester/Mv cholesterol ester (where Δ % cholesterol ester=%

cholesterol ester(enzyme)−% cholesterol ester(control) and Mv cholesterol ester=average molecular weight of the cholesterol esters).

The transferase activity is calculated as a percentage of the total enzymatic activity:

$$\% transferase activity = \frac{A \times 100}{A + Δ \% fatty acid / ( Mv fatty acid )}$$

GLC Analysis

GLC analysis may be performed using any suitable apparatus. In this case a Perkin Elmer Autosystem 9000 Capillary Gas Chromatograph equipped with WCOT fused silica column 12.5 m×0.25 mm ID×.0.1μ film thickness 5% phenyl-methyl-silicone (CP Sil 8 CB from Chrompack) was used.

Carrier gas: Helium.

Injector. PSSI cold split injection (initial temp 50° C. heated to 385° C.), volume 1.0μ³ Detector FID: 395° C.

| | Oven program: | | |
|---|---|---|---|
| | 1 | 2 | 3 |
| Oven temperature, ° C. | 90 | 280 | 350 |
| Isothermal, time, min. | 1 | 0 | 10 |
| Temperature rate, ° C./min. | 1 | 5 | 4 |

Sample preparation: 30 mg of extracted lipid sample was dissolved in 9 ml Heptane:Pyridine, 2:1 containing internal standard heptadecane, 0.5 mg/ml. 300 μl sample solution was transferred to a crimp vial, 300 μl MSTFA (N-Methyl-N-trimethylsilyl-trifluoraceamid) was added and reacted for 20 minutes at 60° C.

Calculation: Response factors for mono-di-triglycerides and free fatty acid were determined from Standard 2 (mono-di-triglyceride), for Cholesterol, Cholesteryl palmitate and Cholesteryl stearate, and the response factors were determined from pure reference material (weighing for pure material 10 mg).

It will be readily apparent to one skilled in the art that compositions comprising lipid acyltransferases for use in the described embodiments may further comprises at least one cholesterol ester as described above.

In one embodiment, the lipid acyltransferase is present in a foodstuff.

The foodstuff may be selected from one or more of: eggs, egg-based products, including but not limited to mayonnaise, salad dressings, sauces, ice creams, egg powder, modified egg yolk and products made therefrom; baked goods, including breads, cakes, sweet dough products, laminated doughs, liquid batters, muffins, doughnuts, biscuits, crackers and cookies; confectionery, including chocolate, candies, caramels, halawa, gums, including sugar free and sugar sweetened gums, bubble gum, soft bubble gum, chewing gum and puddings; frozen products including sorbets, frozen dairy products, including ice cream and ice milk; dairy products, including cheese, butter, milk, coffee cream, whipped cream, custard cream, milk drinks and yoghurts; mousses, whipped vegetable creams, meat products, including processed meat products; edible oils and fats, aerated and non-aerated whipped products, oil-in-water emulsions, water-in-oil emulsions, margarine, shortening and spreads including low fat and very low fat spreads; dressings, mayonnaise, dips, cream based sauces, cream based soups, beverages, spice emulsions and sauces. In certain embodiments, the foodstuff is milk or a milk product.

It will be understood that the methods and uses of the described embodiments can result in a reduced uptake of cholesterol and subsequent lowering of the level of blood cholesterol in an animal. It will be apparent that in cases where the animal has a low starting level of cholesterol in the blood, no or no significant lowering will be seen. However, in animals where the starting level of cholesterol in the blood is high such as in individuals exhibiting hypercholesterolemia, the reduction in the level of cholesterol will be higher. This reduction will be dependent upon the level of cholesterol ester administered with higher levels of cholesterol ester resulting in a greater reduction in cholesterol absorption by the intestinal cells. In various embodiments, the cholesterol ester results in a reduction of at least 2%, 5%, 7%, 10%, 15%, 20%, 25%, 30%, 35%, 40%, 45% or 50% in the uptake of cholesterol by intestinal cells.

### EXAMPLES

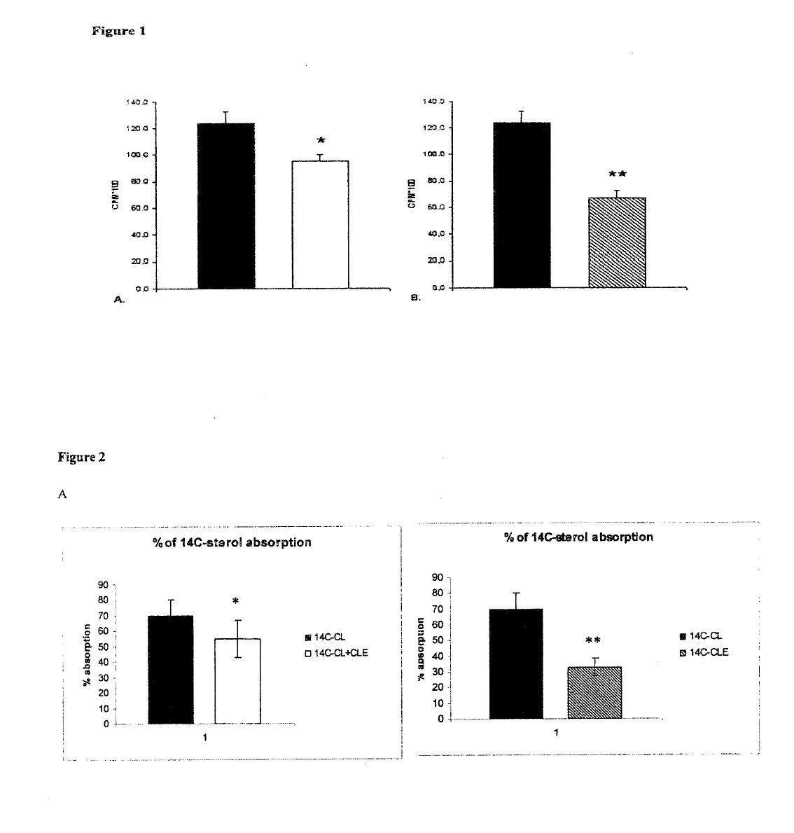

Example 1 and related FIG. 1 shows Sterol absorption into Caco-2 cells using artificial micelles. Panel A: shows inclusion of cholesteryl oleate in cholesterol containing micelles (white column) decreased the uptake of micellar cholesterol into CaCo-2 cells compared to the uptake from micelles containing cholesterol only (black column). Panel B: shows that uptake of cholesteryl. oleate by CaCo-2 cells (hatched column) was less than uptake of cholesterol (black column). Each column and vertical bar represents mean±SD. *p<0.001 for H-cholesterol containing micelles with inclusion of unlabeled cholesteryl oleate, **p<0.001 for 3H-cholesteryl oleate micelles.

Example 2 and related FIG. 2 shows intestinal absorption of sterols in mice using the plasma dual isotope ratio method. Panel A: addition of cholesteryl oleate to cholesterol containing milk (white column) decreased intestinal absorption of cholesterol in mice compared to absorption from milk containing cholesterol only (black column). Panel B: levels of absorption of cholesteryl oleate in the intestines of mice (hatched column) are lower than absorption of cholesterol (black column). Mean±SD. *p<0.05 for addition of cholesteryl oleate to cholesterol containing milk, **p<0.05 for cholesteryl oleate containing milk.

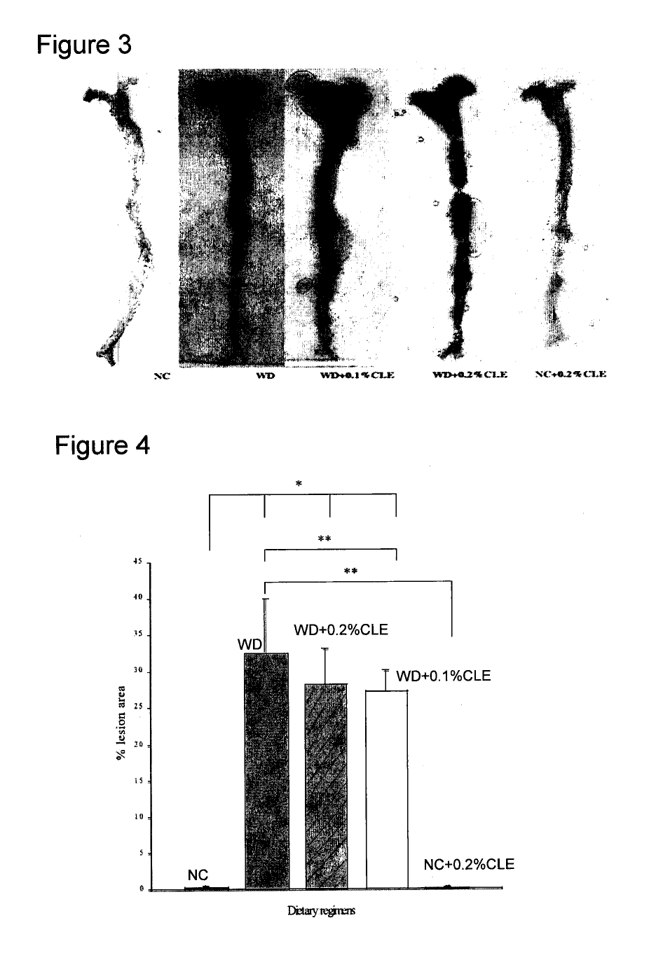

Example 3 and related FIGS. 3 and 4 show the extent and formation of atherosclerotic lesions in mice arteries using ORO staining. FIG. 3 shows digital images of exemplary arteries examined, and FIG. 4 shows experimental results, that mice fed with a diet including an effective amount of a cholesterol ester had reduced total areas of atherosclerotic plaque and lesions.

### Example 1

Materials and Methods

Chemicals

Sodium taurocholate. cholesterol, cholesteryl oleate, oleic acid, phosphatidylcholine in chloroform, sodium dodecyl sulfate (SDS), glucose solution and solvents were purchased from Sigma Aldrich Co. Dulbecco“s modified Eagle's medium (DMEM), PBS, fetal bovine serum (FBS), nonessential amino acids (NEAA), penicillin-streptomycin and tripsin solutions were purchased from Gibco. NUNCLON flasks and 24 well plates were obtained from NUNC. Radiochemicals. [1,2-3H(N)]cholesterol (40-60 Ci/mmol) and cholesteryl oleate [Cholesteryl-1.2-3 H(N)] (30-60 Ci/mmol) were obtained from Perkin Elmer Life Sciences. Opti-Phase Hi Safe 2 liquid scintillant was purchased from Perkin Elmer-Wallac.

Cell Culture

Human colon adenocarcinoma (Caco-2) cells were kindly provided by J T Rasmussen (Department of Molecular Biology Aarhus University C. F. M0llers Alle 3DK-8000 Aarhus CDenmark). Cells were routinely maintained in NUNCLON flasks in Dulbecco's Minimum Essential Medium (DMEM) supplemented with 4.5g/1 glucose. 10% heat-inactivated fetal bovine serum (FBS), 1% nonessential amino acid (NEAA), and 1% antibiotics (complete medium), as previously described (Hidalgo I J. Raub T J. Borchardt R T, Gastroenterology, (1989) ar;96(3):736-49). Once the flasks reached 80% confiuency, the cells were dispersed and seeded into 24-well plates at density 105 cells/well in DMEM supplemented with 10% FBS and 1% NEAA. The cell monolayers were grown to confluence in 37° C. in a humidified atmosphere of 5% C02 in air and allowed to differentiate for 15 days post-confluence with the culture medium replaced every other day.

Preparation of Artificial Micelles

Micelles were prepared according to the method described by Kirana (Kirana C. Rogers P F, Bennett L E, Abevwardena M Y, Patten G S, J Agric Food Chem. (2005) Jun. 1 ; 53(1 1):4623-7) with slight modifications. Briefly, for preparation of micellar solution of H labeled sterols, 14.8 kBq of [1,2-³H] labelled and 0.1 mM unlabeled cholesterol or cholesteryl oleate, respectively, 1 mM oleic acid, 5 mM phosphatidylcholine in chloroform, and 5 mM taurocholate salt were dissolved in ethanol and dried under nitrogen. An equivalent volume of serum-free DMEM was added and the suspension was sonicated three times for 1 min using Branson sonifier cell disrupter. The micelle solution was incubated overnight at 37° C. The solution was then centrifuged at 1000 g for 10 min followed by filtration through a 0.22μm disposable syringe filter (PerkinElmer, Waltham, US-MA). The particle size of micelles was determined by dynamic light scattering (DLS) with use of the Malvern Zetasizer Nano series machine using latex 60 nm Nanosphere Size Standard.

Cholesterol and Cholesteryl Ester Absorption Assay

Differentiated Caco-2 cells were incubated at 37° C. for 1, 3 and 5 hours with micellar solutions containing ³H-labelled cholesteryl oleate according to the method described above. After the incubation step, the cells were lysed with 0.1% (w/v) SDS solution and stored at −20° C. for further analysis. Cell lysates were thawed on ice. After thawing, the lysates were shortly homogenized on ice by a sonifier cell disruptor (60% output, 10 sec) and 1.6 ml of each cell lysate was used for lipid extraction and 2×100 μl was used for direct counting of ³H DPM. Lipids were extracted from the cell lysates according to Bligh and Dyer (1959); briefly, three volumes of methanol:chloroform=2:1 (v/v) were added to 0.8 ml of aqueous samples. To achieve phase separation, 1 volume of water and 1 volume of chloroform were added, and the chloroform phase separated centrifugation was collected and dried under nitrogen. For quantitative analysis ¹⁴C-cholesterol was used as an external standard to correct for the extraction efficiency. Dried lipid fractions were dissolved in chloroform and subjected to TLC (LK5D gel 150) using hexane:diethylether:isopropanol (87:10:3) as mobile phase. The cholesterol and cholesteryl oleate bands were visualized by iodine vapor and scraped off. The lipids were dissolved in hexane, and radioactivity of cholesterol and cholesteryl oleate was measured using a liquid scintillation counter and corrected for extraction efficacy with the ¹⁴C-cholesterol standard.

Monolayers were incubated at 37° C. for 45 min in micellar solutions containing [1,2-³H] cholesterol with or without unlabeled cholesteryl oleate. At the end of the incubation, medium containing micelles was collected and the cells were rinsed twice with cold PBS to remove unincorporated labeled cholesterol. The cells were lysed in 0.1% (w/v) SDS solution. A portion of the cell debris was mixed with Opti-Phase HiSafe 2 scintillant and the radioactivity was determined in a Microbeta Trilux Microplate Scintillation Analyzer (Perkin Elmer-Wallac) to estimate total cholesterol taken up by the cells. To investigate cholesteryl oleate uptake, cells were incubated with micelles containing [1,2-³H] cholesteryl oleate. The cells were analyzed as described above.

Results

Distribution of Micelle Particle Size

In the artificially prepared micelles 100% had a diameter of 63-66 nm (data not shown).

Effect of Cholesteryl Oleate on the Uptake of Micellar Cholesterol

It was postulated that the cholesteryl ester interfered with the uptake of cholesterol from the micelles. To address this, control cells were incubated with micelles containing labeled cholesterol. Another set of cells were incubated with the cholesterol micelles containing unlabeled cholesteryl oleate.

The results show that cells incubated with cholesterol micelles alone accumulated 30% more cholesterol compared to cells incubated with micelles containing both cholesterol and cholesteryl oleate (FIG. 1A). This figure shows that the inclusion of cholesteryl oleate within the micelle decreases the uptake of mi cellar cholesterol by cultured CaCo-2 cells significantly (p<0.001).

Uptake of Micellar Cholesteryl Oleate

To assess whether cholesteryl oleate was absorbed by CaCo-2 cells, cells were incubated with micelles containing either labeled cholesterol or ester.

Compared to cells incubated with micelles containing cholesterol, cells incubated with micelles containing cholesteryl oleate contained approximately 2-fold less labeled sterol (FIG. 1B). This suggests that uptake of the ester, as estimated by cell-associated radiolabeled sterol, was significantly less than that of cholesterol.

Conclusion

Inclusion of cholesteryl oleate in cholesterol containing micelles decreases the uptake of micellar cholesterol by CaCo-2 cells significantly. Furthermore, uptake of cholesteryl oleate by CaCo-2 cells was significantly less than uptake of cholesterol. This indicates that cholesteryl oleate interferes with the uptake of micellar cholesterol. The results suggest that diet enrichment in cholesteryl esters may help to reduce intestinal cholesterol absorption resulting in lower blood cholesterol.

### Example 2

Intestinal Absorption of Cholesterol and Cholesteryl Esters

Material and Method

Plasma Dual Isotope Ratio Method

The net intestinal absorption of cholesterol and cholesteryl esters was measured using the plasma dual isotope ratio method described in Wang et al (Journal of Lipid Research, [2003] 44, 1042-1059).

This method is based on the simultaneous intragastric (IG) and intravenous (IV) administration of [³H] -cholesterol and [¹⁴C] -cholesterol, respectively, and measurement of plasma cholesterol isotope ratios at a set point in time. By definition, the IV [¹⁴C]-cholesterol dose corresponds to “100% absorption”, whereas the [³H]-cholesterol found in the blood reflects the absorption by the gastrointestinal tract. The method allows correction for post-absorptive cholesterol metabolism and for colonic handling of the malabsorbed labelled cholesterol by defining

$$\% Cholesterol absorption = \frac{Percent of IG dose {[}^{14} C ] \text{-} Ch ml plasma}{Percent of IV dose {[}^{3} H ] \text{-} Ch per ml plasma} \times 100 \qquad ( 1 )$$

Experimental Details

Fifteen wild type male mice (C57BL/6J), age of 5 weeks were randomly assigned into 3 groups. An amount of 2.5 μCi of [³H]-cholesterol was dissolved in 100 μl of phosphate buffered saline (PBS) and injected into the tail vein of non fasted and not-anesthetized animals. The animal were then given an oral bolus (IG) dose of either [¹⁴C]-cholesterol, [¹⁴C]-cholesterol+unlabeled cholesteryl oleate (molar ratio 1:1) or [¹⁴C]-cholesteryl oleate dissolved in skimmed milk. Three days later, the mice were anesthetized (pentobarbital, IP) and bled by cardiac puncture into a tube containing heparin. The blood samples were centrifuged to pellet the blood cells and plasma. The percent of cholesterol absorption in plasma was calculated using (1).

Results

As shown in FIG. 2A mice treated with [¹⁴C]-cholesterol and unlabeled cholesteryl oleate (molar ratio 1:1) showed a 12% reduction in cholesterol adsorption compared to [¹⁴C]-cholesterol treated mice (p<0.05).

Furthermore, FIG. 2B shows that [¹⁴C]-cholesteryl oleate treated mice showed a 50% reduction in cholesterol uptake compared to mice treated with [¹⁴C ]-cholesterol. (p<0.001).

Conclusion

Inclusion of cholesteryl oleate in cholesterol containing skimmed milk decreases the absorption of cholesterol by the mouse intestine. Furthermore, the level of intestinal absorption of cholesteryl oleate in mice is lower than absorption of cholesterol. The results indicate that cholesteryl oleate interferes with the absorption of micellar cholesterol. The results suggest that diets enriched in cholesteryl esters can help to reduce intestinal cholesterol absorption.

### Example 3

Reduction of Atherosclerotic Lesions

Material and Method

Quantification of the Percentage of the Surface Covered by Atherosclerotic Lesions

The surface of aorta covered with atherosclerotic lesions was quantified as described by Palinski et al. (1994) and Zampolli et al. (2006). Non-fasted animals were anesthetized with pentobarbital injected intraperitoneally. Next, maximum volume of blood was collected from the heart by direct cardiac puncture of the right ventricle. Blood was kept for further analyses as described above. The heart and arterial tree were then flushed with a heparin-containing cardioplegic solution via a cannula inserted into the left ventricle, followed by perfusion with formalin for 5 minutes. The arterial tree was exposed, branching arteries were removed and entire aorta was dissected from the aortic arch down to the iliac bifurcation. Using a stereomicroscope, aortas were cleaned from periadventitial tissue; remaining branches were cut off and opened longitudinally by incision following the inner curvature of the aortic arch and the ventral side of the aorta. Additional incisions followed the outer curvature of the arch to the subclavian artery. An Oil Red 0 (ORO) staining solution was prepared by diluting the ORO stock solution (5 mg/ml in 2-propanol; Sigma Aldrich) with deionized water (6:4). Dissected aortas were stained for 30 minutes with ORO, followed by rinsing with 2-propanol and deionized water. Stained aortas were then laid flat on the microscope slide with endothelial side facing upwards. The tissues were mounted with aqueous mounting agent (Aquatex®, Merck Chemicals, Darmstadt, Germany) and covered with glass slide cover slip.

Slide Imaging and Computer Assisted Analysis

Digital images of the slides were obtained through the 48-bit color scanner (Canoscan 9950F, Canon) with 1200 DPI resolution. The images were captured in Photoshop CS3 Extended software and converted to gray scale. After further processing, including sharpness, contrast and brightness adjustments, and the image of aorta was printed out in two copies. Aorta outline was drawn by the observer with a red pen on the one copy and lesion outlines were drawn on the other one. Drawings were done blindly by the observer. Images of a hand-drawn aorta outlines and regions (patches) matching atherosclerotic lesions were then scanned and re-imported into Adobe Photoshop and the images were then evaluated obtain the percent of aorta covered with atherosclerotic lesions. Briefly, in the digitized image of entire aorta and the image of the patches corresponding to lesions, red outlined areas were converted to black and the blackness values for all pixels in the image were summed and counted.

Experimental Details

Transgenic mice were randomized into 5 dietary regiments: (1) Normal chow (NC), (2) NC+0.2% cholesterol oleate (CLE), (3) Western type diet (WD) containing 0.2% cholesterol, (4) WD containing 0.2% cholesterol and 0.2% CLE, (5) WD containing 0.1% cholesterol and 0.1% CLE. The extent of the lesions was quantified as percent area positive to lipid staining by ORO in the entire aorta after 28 weeks of experimental treatments.

Results

As shown in FIGS. 3 and 4, in mice fed with NC and NC+0.2% CLE for 28 weeks, en face ORO staining showed only marginal atherosclerotic plaques formation (total plaque areas of 1±0.7% and 1.2±0.9% respectively) and there was no difference between those two treatments. Feeding WD, WD+0.2% CLE, and WD+0.1% CLE showed that CLE lowered atherosclerotic lesions.

Conclusion

The results indicate that cholesteryl oleate interferes with the absorption of micellar cholesterol and provides a reduction in atherosclerotic lesions. Diets enriched in cholesteryl esters reduce atherosclerotic lesions, although the benefit is, surprisingly, not directly related to the amount of cholesteryl ester enrichment. For example, mice fed with WD+0.2% CLE showed higher rates of atherosclerosis than mice fed with WD+0.1% CLE. Although the exact mechanism for the non-direct relationship is presently unknown, at least one current hypothesis holds that cholesterol may be derived from cholesteryl esters by hydrolysis of the excess cholesteryl esters within the digestive system. At least one supporting hypothesis holds that the rate of hydrolysis is dependent on the activity of pancreactic carboxylester lipase (CEL), which catalyzes the hydrolysis of dietary cholesteryl esters (Hui, D Y, Molecular biology of enzymes involved with cholesterol ester hydrolysis in mammalian tissues, Biochim Biphys Acta. 1996). The rates of absorption of different cholesteryl esters are correlated to their hydrolysis by CEL (Rudd, E A and Brockman, H L, Lipase (Borgstrom B & Brockman H L, Eds.) 1984, Elsevier Science Publishers, Amsterdam). Accordingly, CEL and/or the presence of other enzymes within the digestive system may be responsible for a maximum benefit related to cholesteryl ester enrichment, beyond which increasing the concentration of cholesteryl esters causes the equilibrium to shift in favor of hydrolyzing cholesteryl esters to free cholesterol which is absorbed by the digestive tract. The current embodiments therefore include, in part, the discovery that reduced dietary CLE supplement levels, for example 0.1% versus 0.2%, may reduce atherosclerotic lesions in animals.

All publications mentioned in the above specification are herein incorporated by reference. Various modifications and variations of the described methods and system of the described embodiments will be apparent to those skilled in the art without departing from the scope and spirit of the described embodiments. Although the described embodiments have been described in connection with specific embodiments, it should be understood that the embodiments as claimed should not be unduly limited to such specific embodiments.

Indeed, various modifications of the described modes for carrying out the embodiments which are obvious to those skilled in biochemistry and biotechnology or related fields are intended to be within the scope of the following claims.

What is claimed is:

1. A method for reducing atherosclerotic lesions in an animal comprising administering to the animal a composition comprising an effective amount of at least one cholesterol ester to reduce atherosclerotic lesions.

2. The method according to claim 1 wherein the cholesterol ester is a cholesterol fatty acid ester, and the fatty acid is a C4 to C24 fatty acid.

3. The method according to claim 1, wherein the cholesterol ester comprises at least one of cholesteryl linoleate (C18:2), cholesteryl oleate (C18:1), cholesteryl stearate (C18:0), cholesteryl palmitate (C16:0), cholesteryl palmitoleate (C16:1), cholesteryl myristate (C14:0), cholesteryl laurate (C12:0) or cholesteryl caprate (C10:0).

4. The method according to claim 1, wherein the cholesterol ester is produced from at least one of egg, milk or meat.

5. The method according to claim 1, wherein the cholesterol ester is administered at a dosage of between about 0.001 g and about 10 g per day.

6. The method of claim 5, wherein the cholesterol ester is administered at a dosage of between about 0.01 g and about 5 g per day.

7. The method of claim 6, wherein the cholesterol ester is administered at a dosage of between about 0.1 g and about 3 g per day.

8. The method according to claim 1, wherein the cholesterol ester is present in a foodstuff.

9. The method according to claim 8, wherein the foodstuff is selected from one or more of: eggs, egg-based products, including but not limited to mayonnaise, salad dressings, sauces, ice creams, egg powder, modified egg yolk and products made therefrom; baked goods, including breads, cakes, sweet dough products, laminated doughs, liquid batters, muffins, doughnuts, biscuits, crackers and cookies; confectionery, including chocolate, candies, caramels, halawa, gums, including sugar free and sugar sweetened gums, bubble gum, soft bubble gum, chewing gum and puddings; frozen products including sorbets, frozen dairy products, including ice cream and ice milk; dairy products, including cheese, butter, milk, coffee cream, whipped cream, custard cream, milk drinks and yoghurts; mousses, whipped vegetable creams, meat products, including processed meat products; edible oils and fats, aerated and non-aerated whipped products, oil-in-water emulsions, water-in-oil emulsions, margarine, shortening and spreads including low fat and very low fat spreads; dressings, mayonnaise, dips, cream based sauces, cream based soups, beverages, spice emulsions and sauces.

10. The method according to claim 8, wherein the cholesterol ester is added to the foodstuff or is produced in situ.

11. An atherosclerotic lesion reducing composition comprising an effective amount of at least one cholesterol ester.

12. The composition according to claim 11, wherein the cholesterol ester is a cholesterol fatty acid ester and the fatty acid is a C4 to C25 fatty acid.

13. The composition according to claim 11, wherein the cholesterol ester comprises at least one of cholesteryl linoleate (C18:2), cholesteryl oleate (C18:1), cholesteryl stearate (C18:0), cholesteryl palmitate (C16:0), cholesteryl palmitoleate (C16:1), cholesteryl myristate (C14:0), cholesteryl laurate (C 12:0) or cholesteryl caprate (C 10:0).

14. The composition according to claim 11, wherein the cholesterol ester is produced from at least one of egg, milk or meat.

15. The composition according to claim 11, wherein the composition comprises a foodstuff.

16. The composition according to claim 15, wherein the foodstuff is selected from selected from one or more of: eggs, egg-based products, including but not limited to mayonnaise, salad dressings, sauces, ice creams, egg powder, modified egg yolk and products made therefrom; baked goods, including breads, cakes, sweet dough products, laminated doughs, liquid batters, muffins, doughnuts, biscuits, crackers and cookies; confectionery, including chocolate, candies, caramels, halawa, gums, including sugar free and sugar sweetened gums, bubble gum, soft bubble gum, chewing gum and puddings; frozen products including sorbets, frozen dairy products, including ice cream and ice milk; dairy products, including cheese, butter, milk, coffee cream, whipped cream, custard cream, milk drinks and yoghurts; mousses, whipped vegetable creams, meat products, including processed meat products; edible oils and fats, aerated and non-aerated whipped products, oil-in-water emulsions, water-in-oil emulsions, margarine, shortening and spreads including low fat and very low fat spreads; dressings, mayonnaise, dips, cream based sauces, cream based soups, beverages, spice emulsions and sauces.

17. The composition according to claim 15, wherein the cholesterol ester is added to the foodstuff or is produced in situ.

18. A pharmaceutical composition comprising an effective amount of at least one cholesterol ester for use in reducing atherosclerotic lesions in an individual, and at least one of a pharmaceutically acceptable diluent, excipient, or carrier.

19. The pharmaceutical composition of claim 18 further comprising at least one lipid acyltransferase.

20. The pharmaceutical composition according to claim 19, wherein the lipid acyltransferase is classified as an enzyme of class E.C. 2.3.1.x.

21. The pharmaceutical composition according to claim 20, wherein the enzyme has at least 5% activity.

22. The pharmaceutical composition according to claim 18, wherein the cholesterol, ester is at least one of a cholesterol fatty acid ester wherein the fatty acid is a C4 to C24 fatty acid.

23. The pharmaceutical composition according to claim 18, wherein the cholesterol ester comprises at least one of cholesteryl linoleate (C18:2), cholesteryl oleate (C18:1), cholesteryl stearate (C 18:0), cholesteryl palmitate (C16:0), cholesteryl palmitoleate (C16:1), cholesteryl myristate (C14:0), cholesteryl laurate (C12:0) or cholesteryl caprate (C10:0).
